(19) United States
(12) Patent Application Publication
(10) Pub. No.: US 2003/0129142 A1
(43) Pub. Date: Jul. 10, 2003

(54) Underarm product and package combination which redefines the consumers' habits and practices

(76) Inventors: Kenneth Michael Schroeder, Erlanger, KY (US); Scott Edward Smith, Cincinnati, OH (US); Theresa Louise Johnson, Betesville, IN (US); Melissa Ann Hallmark, Cincinnati, OH (US)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 10/285,918

(22) Filed: Nov. 1, 2002

Related U.S. Application Data
(60) Provisional application No. 60/332,855, filed on Nov. 6, 2001.

(51) Int. Cl. A61K 9/00; B65D 88/54

(52) U.S. Cl. 424/47; 222/320

(57) ABSTRACT

An underarm product and package combination including an underarm product, said underarm product being a liquid, said underarm product being clear and a package, said package capable of containing and dispensing said underarm product, said package having an overflow volume of no greater than 118 milliters, said package being clear. The package may be made of plastic. The underarm product may be a single-phase or multi-phase solution. The underarm product may be free of suspended or dispersed solids. The package may be a contact or non-contact type dispenser. The package may have a single or multiple layer wall.

### CROSS REFERENCE TO RELATED APPLICATION

This application claims the benefit under 35 USC 119(e) to U.S. application Ser. No. 60/332,855, filed Nov. 6, 2001.

### TECHNICAL FIELD

The present invention relates to an underarm product in combination with a package which redefines the consumers' habits and practices in a way that makes carrying and using the product in front of other people more socially acceptable, thus increasing the chance of re-application of the product throughout the day.

### BACKGROUND OF THE INVENTION

The consumer products industry provides the world's consumers with a wide variety of personal care products that are designed to meet consumer's needs. These personal care products are designed to not only meet the functional needs of consumers but also create a usage experience that is pleasurable. The number and variety of personal care products that are available to today's women consumers is vast and spans a broad range of functional design, aesthetic design, and intended use. These personal care products can be grouped in numerous ways. For example, products can be grouped by function (cleansing, prevention, treatment, cosmetic enhancement, sensory experience, etc.), form (sprays, creams, lotions, wipes, bars, lathering soaps, etc.), and/or intended use (for hair, teeth, facial skin, legs, underarms, whole body).

It has been discovered through qualitative and quantitative consumer studies that there are specific, measurable habits and practices associated with each of these personal care products. These habits and practices include frequency of use, usage occasion, product storage, multiple-use, combination use, and choices for brand, form, scent, price, and retail environment. An example of this is a consumer's habit or practice to carry certain of these personal care products with them and/or their habit or practice to use the product in front of others. Certain cosmetics, for example, are very frequently carried by users and often, comfortably used in front of others. Examples of this include lipstick and compact pressed powder. These types of personal care products are commonly re-applied multiple times during the day and are commonly re-applied in front of other people. Another example of this is perfume or cologne, which is also often carried by the user, re-applied during the day, and used in front of others.

There are other types of personal care products, such as underarm products, that while re-application may be desirable, cannot be achieved easily, conveniently, or with sufficient desired discretion. Many consumers would like to “freshen up” throughout the day and apply/re-apply their underarm product. This re-application refreshes them, gives a fresh burst of fragrance and renews their confidence. Some consumers already have such re-application habits but must store their product in inconvenient locations (desk drawer, locker, gym bag) and interrupt their daily routine to do the application/re-application in a discreet location (restroom, locker room, home). For example, canister-type packages are messy and unsightly while spray type underarm products are currently packaged predominantly in large, bulky, unsightly aerosol cans. These types of packaging do not allow the user to conveniently carry the product with them in their purse, nor are they designed in such a way to be acceptable to use in front of others. Even if smaller sizes were made available, the metal can packaging is easily associated with the intended use of underarm products and therefore discourages the user from carrying the product or using the product as often as they want based on need.

### SUMMARY OF THE INVENTION

The present invention provides an underarm product and package combination including an underarm product, said underarm product being a liquid, said underarm product being clear and a package, said package capable of containing and dispensing said underarm product, said package having an overflow volume of no greater than 118 milliters, said package being clear. The package may be made of plastic. The underarm product may be a single-phase or multi-phase solution. The underarm product may be free of suspended or dispersed solids. The package may be a contact or non-contact type dispenser. The package may have a single or multiple layer wall.

Other advantages and novel features of the present invention will become apparent to those skilled in the art from the following detailed description, which simply illustrates various modes contemplated for carrying out the invention. As will be realized, the invention is capable of other different obvious aspects, all without departing from the invention. Accordingly, the drawings and descriptions are illustrative in nature and not restrictive.

### BRIEF DESCRIPTION OF THE DRAWINGS

While the specification concludes with claims which particularly point out and distinctly claim the present invention, it is believed that the present invention will be better understood from the following description of preferred embodiments, taken in conjunction with the accompanying drawings, in which like reference numerals identify like elements and wherein:

FIG. 1 is a plan view of a package in accordance with the present invention;

FIG. 2 is a plan view of the package in FIG. 1 having an overcap;

FIG. 3 is a right side elevational view of the package in FIG. 2;

FIG. 4 is a front elevational view of the package in FIG. 2;

FIG. 5 is a left side elevational view of the package in FIG. 2;

FIG. 6 is a top view of the package in FIG. 2;

FIG. 7 is a plan view of another package in accordance with the present invention;

FIG. 8 is a plan view of the package in FIG. 7 having an overcap;

FIG. 9 is a right side elevational view of the package in FIG. 8;

FIG. 10 is a front elevational view of the package in FIG. 8;

FIG. 11 is a left side elevational view of the package in FIG. 8;

FIG. 12 is a top view of the package in FIG. 8;

FIG. 13 is a plan view of another package in accordance with the present invention;

FIG. 14 is a plan view of the package in FIG. 13 having an overcap;

FIG. 15 is a right side elevational view of the package in FIG. 14;

FIG. 16 is a front elevational view of the package in FIG. 14;

FIG. 17 is a left side elevational view of the package in FIG. 14;

FIG. 18 is a top view of the package in FIG. 14;

FIG. 19 is a plan view of another package in accordance with the present invention;

FIG. 20 is a plan view of the package in FIG. 19 having an overcap;

FIG. 21 is a right side elevational view of the package in FIG. 20;

FIG. 22 is a front elevational view of the package in FIG. 20;

FIG. 23 is a left side elevational view of the package in FIG. 20;

FIG. 24 is a top view of the package in FIG. 20;

FIG. 25 is a plan view of another package in accordance with the present invention;

FIG. 26 is a right side elevational view of the package in FIG. 25;

FIG. 27 is a front elevational view of the package in FIG. 25;

FIG. 28 is a left side elevational view of the package in FIG. 25;

FIG. 29 is a top view of the package in FIG. 25;

FIG. 30 is a plan view of another package in accordance with the present invention;

FIG. 31 is another plan of the package in FIG. 30;

FIG. 32 is a cross-sectional view of another package in accordance with the present invention; and

FIG. 33 is a cross-sectional view of another package similar to FIG. 32 having multilayers.

### DETAILED DESCRIPTION OF THE INVENTION

It has recently been discovered that it is possible to design an underarm product (defined herein as a product which provides or is intended to provide antiperspirant and/or deodorant efficacy) in such a way that it re-characterizes or reframes the perceptions of the product. Specifically, the perceptions of convenience, ease of re-application, willingness to carry and use product in front of others can be altered. These perceptions can be altered with package size, shape, color, and/or materials of construction. In the case of an underarm product, consumers' perceptions can be dramatically altered by choosing package size, shape, color and/or material that would be typically associated with products such as perfume/cologne and body spray that consumer's comfortably carry with them and use in front of other people. When we reframe the perceptions of an underarm product in this way, consumers will not only be more willing to carry the product with them and use in front of other people, but will likely increase the frequency of product use and improve antiperspirancy/deodorancy benefits.

A consumer test was conducted to prove our hypothesis and designs. First, we discovered that consumers' willingness to carry personal care products with them is dependant upon what product type the consumers perceive the product to be. For example, consumers prefer to carry skin care products over perfumes/colognes, perfumes/colognes over body spray/mist, and body spray/mist over underarm product. Secondly, we discovered that consumers' willingness to use the personal care products in front of other people is also dependant upon what product type the consumers perceive the product to be. For example, consumers prefer to use perfume/cologne over skin care products, skin care products over body spray/mist, and body spray/mist over underarm product.

Referring to Table A below, we showed a set of two empty packages to consumers. The first package was a standard aluminum can. The second package was a clear plastic bottle. We then asked consumers what type of product would you expect to see in these two packages. The large majority of consumers (78%) perceive that the aluminum can would contain an underarm product. While 72% of the consumers perceived that the clear plastic package would contain a perfume/cologne type product. Interestingly, 0% of the consumers thought that the clear plastic bottle would contain an underarm product.

TABLE A

| | | Clear plastic | |
|---|---|---|---|
| | Aluminum Can | bottle | Neither |
| Perfume | 14% | 72% | 14% |
| Bad Spray | 59% | 38% | 3% |
| Underarm product | 78% | 0% | 22% |
| Skin Care | 5% | 64% | 31 |

The term “plastic” is defined herein as any substance or material that is capable of being shaped or molded, with or without the application of heat. Usually plastics are a high homo-polymer or co-polymer that are mostly thermoplastic or thermosetting of high molecular weight. Plastics fitting this definition include, but are not limited to, polyolefins, polyesters, nylon, vinyl, acrylic, polycarbonates, polystyrene, and polyeurothane.

The term “clear” is defined herein as having the property of transmitting light without appreciable scattering so that bodies lying behind are perceivable. One acceptable test method for determining whether a product is clear is to attempt to read a series of words placed immediately behind the package. The words being printed in black color, 14 point Times New Roman font, printed on a white sheet of paper with the printed side of the paper attached to the back of the package. The word and/or letters must be visible and/or readable from the front of the package ny an individual of reasonable eye sight and positioned directly in front of the package.

Referring to Table B below, we showed a set of clear plastic bottles containing product to consumers. The first bottle contained a colorless liquid. The second bottle contained a colorless liquid having undissolved powder (e.g., conventional underarm product powder). We then asked consumers which package do you believe represents each of the following product types. When asked which package contained perfume, the vast majority of consumers (94%) consider that a perfume/cologne type product would look like a clear liquid in a clear package. Interestingly, 0% of the consumers considered perfume to contain any powdery substance. When asked which package contained underarm product, only 8% of the consumers considered the clear plastic package containing a clear colorless liquid to contain an underarm product. Even further, when asked which package contained a skin care product, 62% of the consumers selected the package containing a powdery substance. This trend seems to indicate that consumers believe the presence of a powdery substance (i.e., not just a colorless liquid) is more efficacious at treating the skin as is commonly considered the functions of an underarm product or skin care product, more so than a perfume or body spray.

TABLE B

| | Clear plastic bottle | Clear plastic bottle | |
|---|---|---|---|
| | Clear, colorless | Liquid/powder contents | |
| | liquid | of AP | Neither |
| Perfume | 94% | 0% | 6% |
| Body Spray | 84% | 11% | 5% |
| Underarm | 8% | 39% | 53% |
| product | | | |
| Skin Care | 8% | 62% | 30% |

Referring to Table C below, we showed a set of clear plastic bottles containing clear product to consumers. The first bottle is substantially similar to the second bottle except it is substantially smaller in size. We then asked consumers which package do you believe represents each of the following product types. When asked which package contained perfume, the vast majority of consumers (89%) consider that a perfume/cologne type product would be stored in the smaller bottle. Amazingly, when asked which package contained underarm product, 0% of the consumers selected the smaller package.

TABLE C

| | Clear plastic bottle | Large Size | |
|---|---|---|---|
| | Clear, colorless | Clear Plastic bottle, | |
| | liquid | Clear Colorless liquid | Neither |
| Perfume | 89% | 6% | 6% |
| Body Spray | 43% | 54% | 3% |
| Underarm | 0% | 47% | 53% |
| product | | | |
| Skin Care | 25% | 28% | 47% |

### PACKAGE EXAMPLES

The following examples and embodiments of clear plastic packages illustrate dispensing packages within the scope of the present invention. The examples are given solely for the purpose of illustration and are not constructed as limitations of the present invention, as many variations thereof are possible without departing from the spirit of the invention.

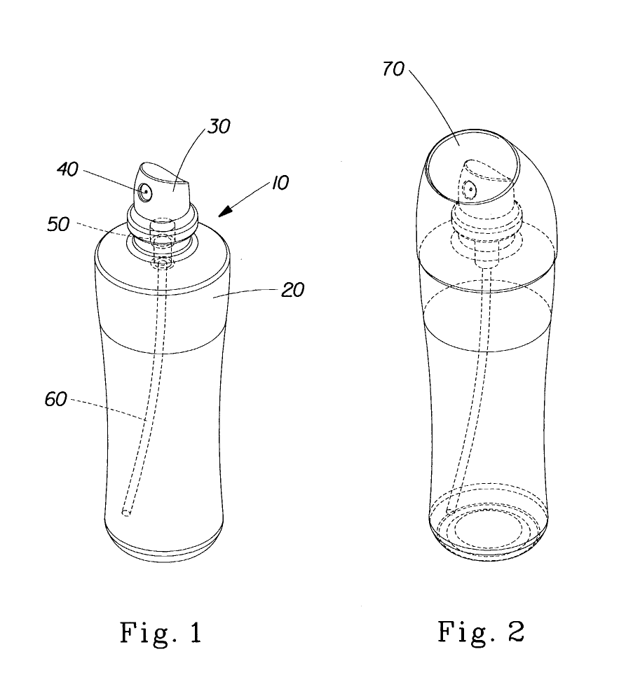

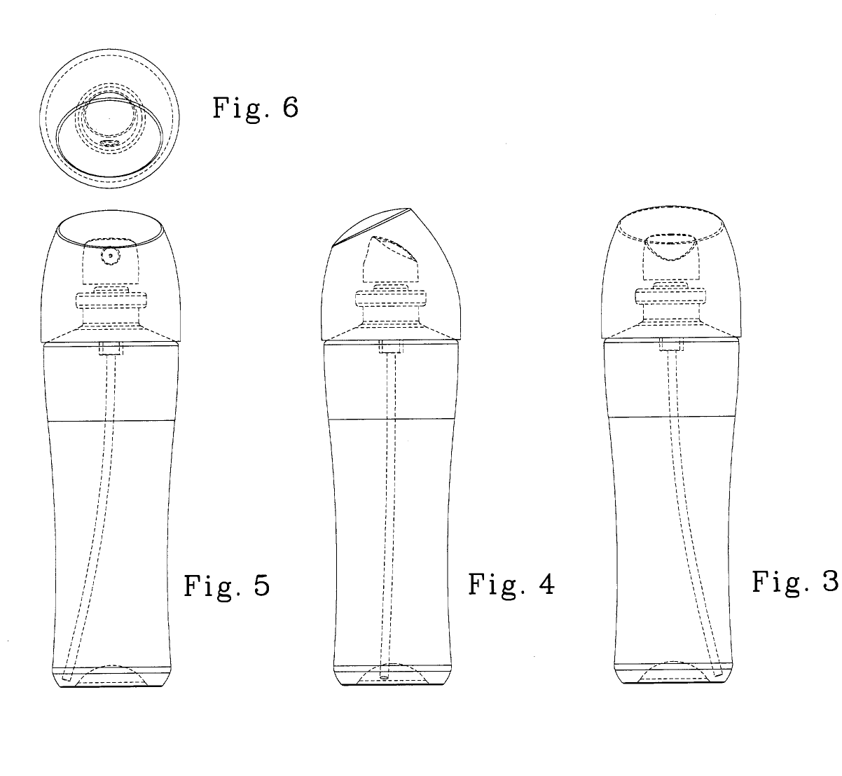

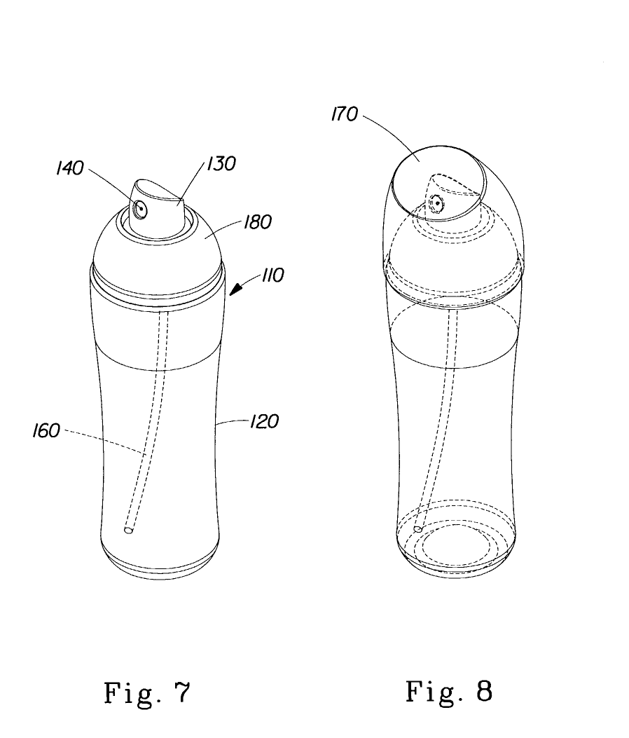

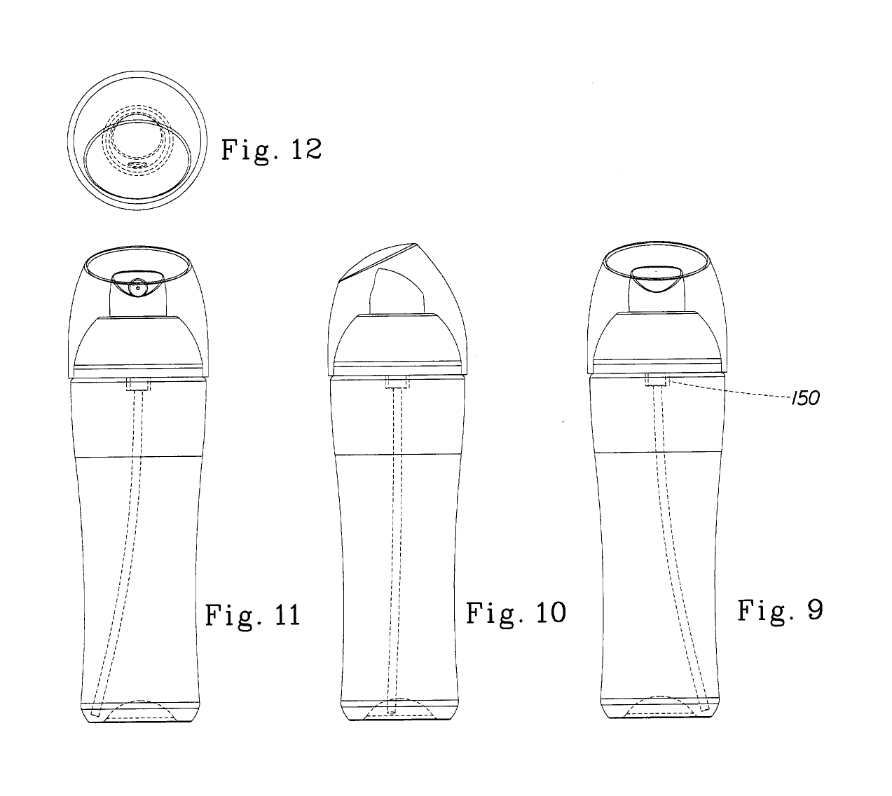

The examples shown in FIGS. 1-31 depict clear plastic packages suited for delivering clear underarm product. FIG. 1 illustrates a package 10, a container body 20 (suitable for holding pressure) designed to have a valve (or suitable closure) affixed to its opening, an actuator 30, an orifice opening 40, a valve 50, a diptube 60 and in FIG. 2 an overcap 70. FIG. 3-6 show additional views of the package in FIG. 1.

FIG. 7 illustrates a package 110, a container body 120 (suitable for holding pressure) designed to have a valve (or suitable closure) affixed to its opening, an actuator 130, an orifice opening 140, a diptube 160, a collar 180 and in FIG. 8 an overcap 170, while FIG. 9 shows a valve 150. FIG. 10-12 show additional views of the package in FIG. 7.

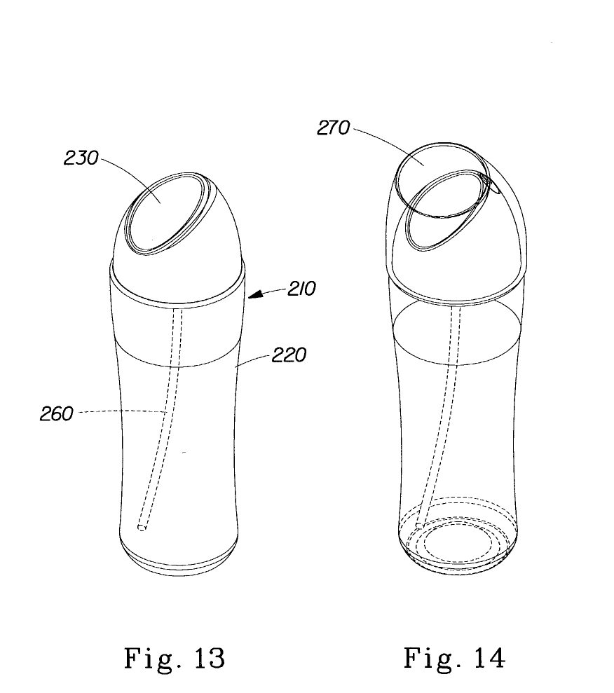

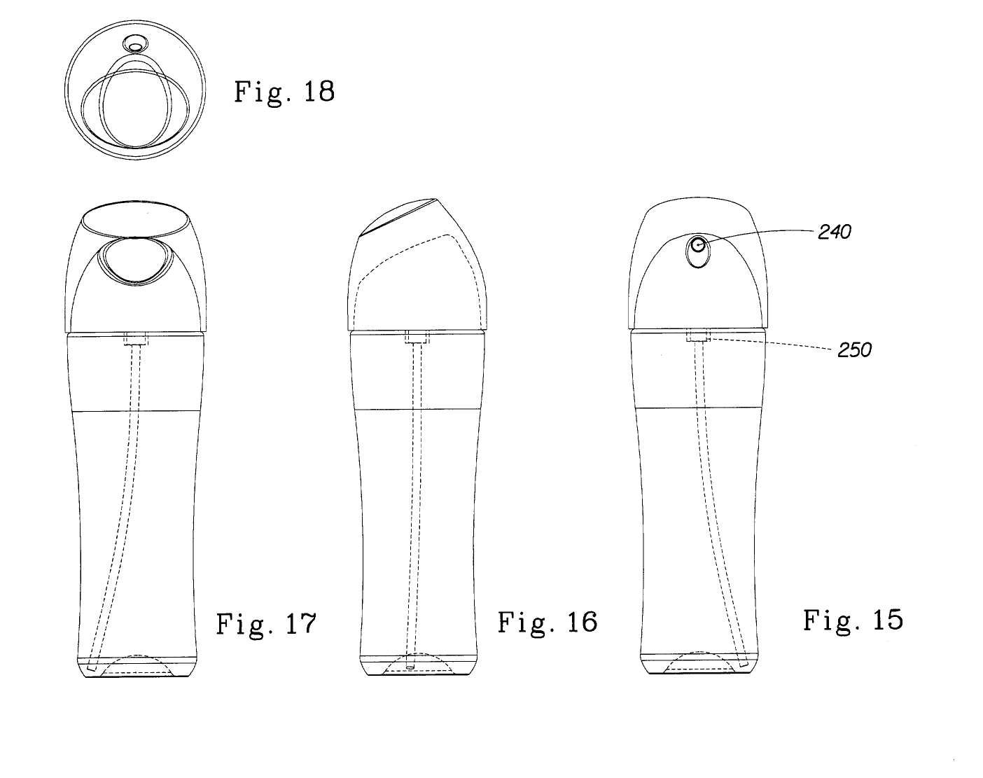

FIG. 13 illustrates a package 210, a container body 220 (suitable for holding pressure) designed to have a valve (or suitable closure) affixed to its opening, an actuator 230, a diptube 260 and in FIG. 14 an overcap 270, while FIG. 15 shows a valve 250 and an orifice opening 240. FIG. 16-18 show additional views of the package in FIG. 13.

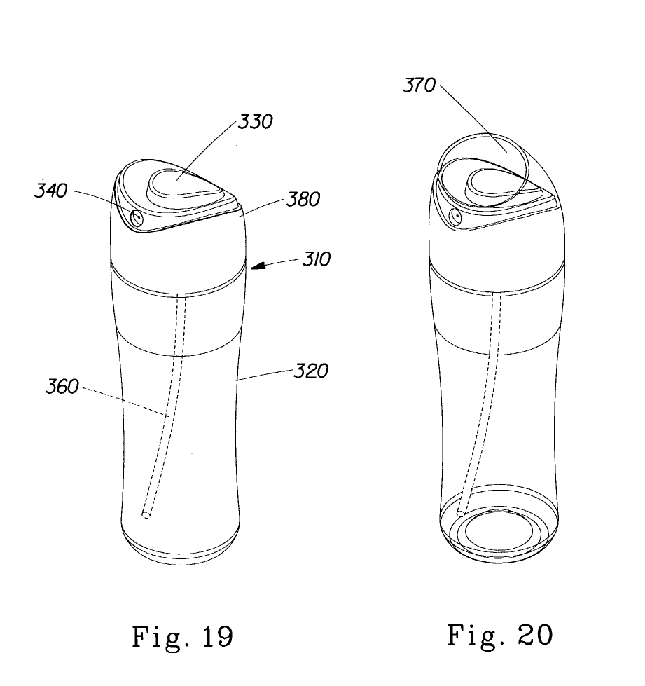

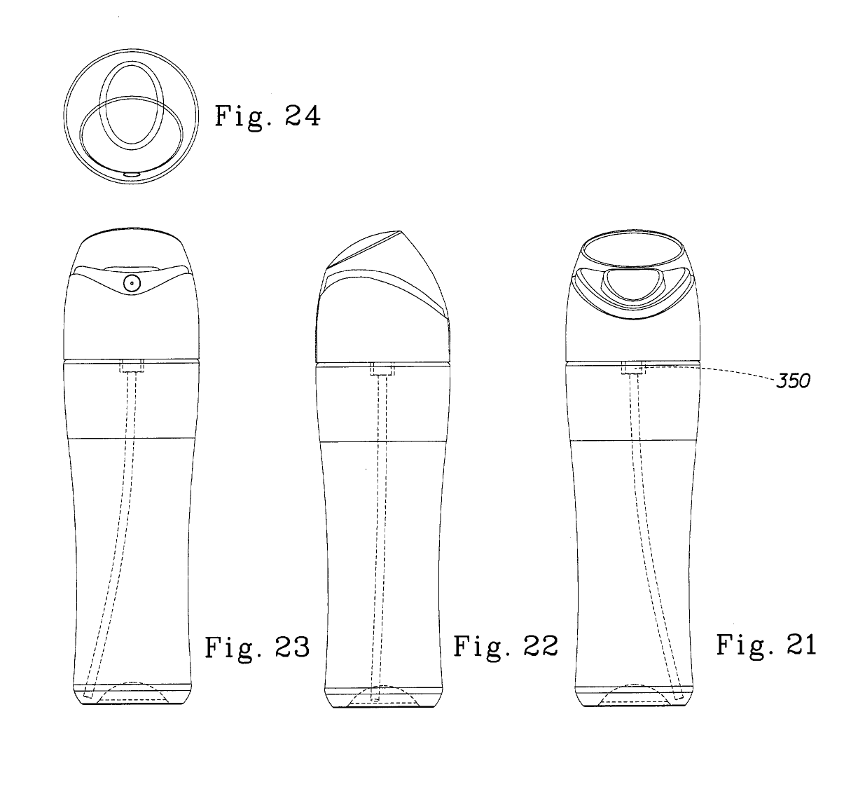

FIG. 19 illustrates a package 310, a container body 320 (suitable for holding pressure) designed to have a valve (or suitable closure) affixed to its opening, an actuator 330, a diptube 360, an orifice opening 340, a collar 380 and in FIG. 20 an overcap 370, while FIG. 21 shows a valve 250. FIGS. 22-24 show additional views of the package in FIG. 19.

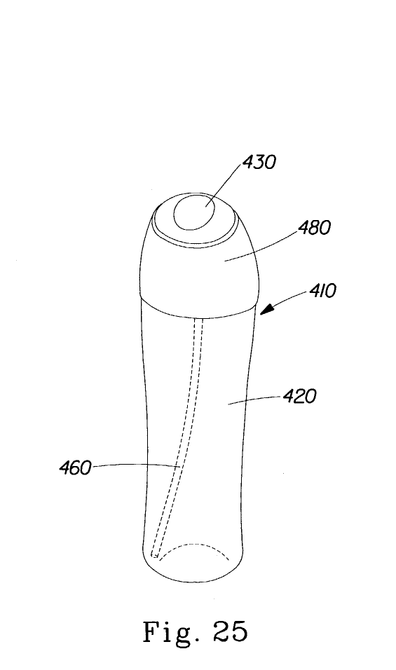

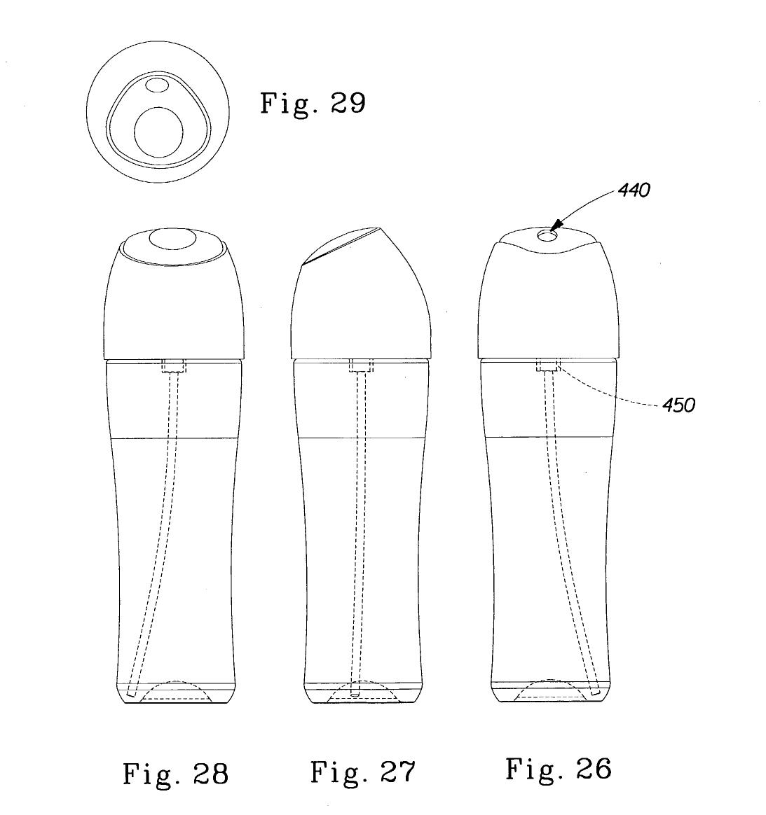

FIG. 25 illustrates a package 410, a container body 420 (suitable for holding pressure) designed to have a valve (or suitable closure) affixed to its opening, an actuator 430, a diptube 460, a collar 480 and FIG. 26 shows a valve 450 and an orifice opening 440. FIGS. 27-29 show additional views of the package in FIG. 19.

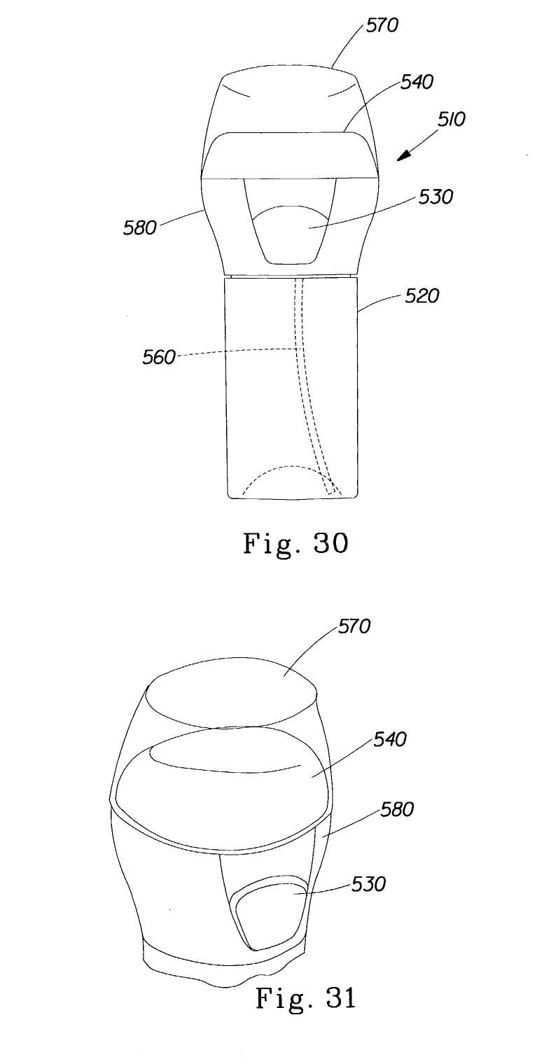

FIG. 30 illustrates a package 510, a container body 520 (suitable for holding pressure) designed to have a valve (or suitable closure) affixed to its opening, an actuator 530, a diptube 560, a shroud 580, a valve (not shown), an application surface 540 having at least one orifice opening (not shown), and an overcap 570. FIG. 31 shows an additional view of the package in FIG. 30.

The package of the present invention may be designed to be a contact (e.g., FIGS. 1-29) or non-contact (e.g., FIGS. 30-31) type dispenser. The contact type package may have an application surface which may come into direct contact with the application target (e.g., consumer's skin). The non-contact package may be designed to spray, or other like projection methods, the underarm product to the application target (e.g., consumer's skin).

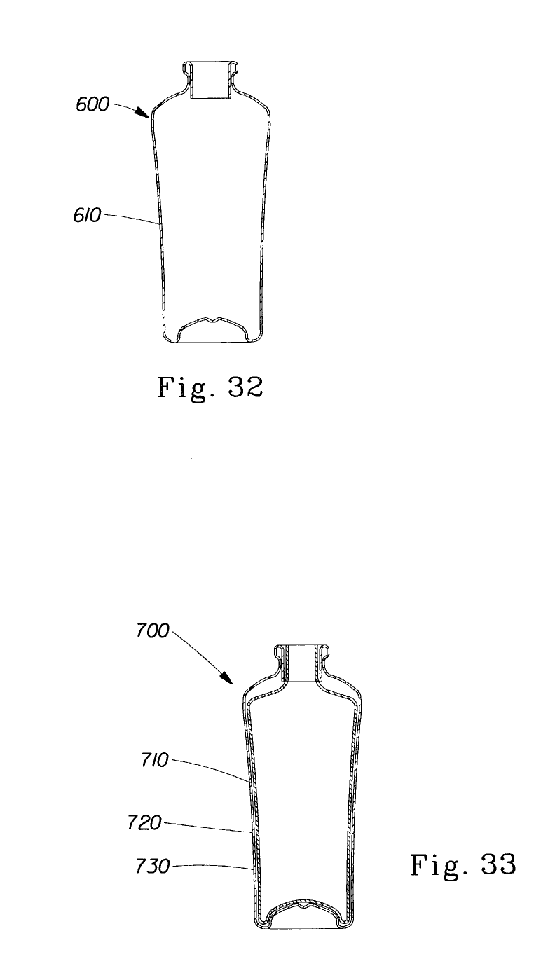

FIGS. 32 and 33 illustrate the dimensions of a package suitable for the containment of underarm product. The package has an overflow volume no greater than 118 milliters.

A package of the present invention may be produced by any mode of package formation including, but not limited to, injection blow molding, injection stretch blow molding, injection molding or extrusion molding. Clear resins with suitable plastics chemical and mechanical properties could be molded to the dimensions shown in FIGS. 30 and 31. In FIG. 30, the package 600 is shown with a mono-wall 610 having a thickness greater than 0.025″ in wall thickness. The package can be produced of any clear plastic having suitable mechanical and chemical properties such as polyethylene teraphthalate (PET) supplied by GE Plastics 9921, Hypertuf 86017 supplied by M&G Polymers USA, Barex supplied by BP USA, and /or Nylon G21 supplied by Grivory Ems. In FIG. 31, package 700 is shown having multiple walls 710, 720, 730. FIG. 31 could utilize multiple plastics having a variety of thickness and/or combining multiple materials that maintain its clear property. The materials used in FIG. 31 could provide additional mechanical or chemical properties.

### UNDERARM PRODUCT EXAMPLES

The following examples and embodiments of clear plastic packages illustrate dispensing packages within the scope of the present invention. The examples are given solely for the purpose of illustration and are not constructed as limitations of the present invention, as many variations thereof are possible without departing from the spirit of the invention.

The packages described herein are designed to deliver products that are clear liquids, which reduce odor and/or wetness in the axilla. These products typically do not include any powders such as particulate antiperspirant actives, talc, clays, etc., that reduce the clear nature of the product. These liquid products can exist in a variety of forms including single-phase solutions and multiphase solutions. Products based on multiphase solutions can either be clear stable emulsions or two clear immiscible layers that must be shaken by the consumer prior to application (this product may or may not be clear upon shaking).

Underarm products used in the present invention may include any clear liquid product known in the antiperspirant and/or deodorant art. Typically these product include a propellant such as butane, isobutane, propane, dimethyl ether, 1,1 difloroethane or mixtures thereof. The products also include a liquid phase that can be composed on any solvent known to the cosmetic field including: water, ethanol, cyclopentasiloxane, dimethicone, propylene glycol or other similar solvents. These products also typically comprise a deodorant active which is soluble in the liquid product. Typical actives include triclosoan, triclorocarban, zinc phenol sulfate, or any other known deodorant active. These products also typically contain a fragrance that is effective at masking malodor associated with perspiration or which otherwise provide the composition with the desired perfume aroma. Examples of useful deodorant actives and fragrances are included in U.S. Pat. No. 5,902,572.

Underarm product of the present invention may also include a solubilized antiperspirant active. Typically these actives are astrigent salts or aluminum, zirconium or zinc but any known antiperspirant active can be employed including, but not limited to, aluminum chlorohydrate, aluminum sesquichlorohydrate, aluminum zirconium trichlorhydrate, and aluminum zirconium tretrachlorhydrate. The active can be solubilized in any solvent capable of dissolving the active, these can include, but are not limited to, ethanol, water, propylene glycol, glycerin, sorbitol, 1,2 hexanediol, 1,2 pentanediol, and 1,2 butanediol. These products also typically contain cosmetics solvents such as cyclomethicone, dimethicone, mineral oil, dimethicone copolyol, demethiconol or other solvent that provides a desirable skin feel. These products can also include fragrance, deodorant actives, or skin moisturizers. Examples of such products are disclosed in WO 96/18378, U.S. Pat. No. 4,073,880; U.S. Pat. No. 5,814,309, U.S. Pat. No. 3,555,145 and co-pending and co-owned U.S. patent application Ser. No. 09/864012.

Having shown and described various embodiments of the present invention, further adaptations of the of the present invention as described herein can be accomplished by appropriate modifications by one of ordinary skill in the art without departing from the scope of the present invention. Several of these potential modifications and alternatives have been mentioned, and others will be apparent to those skilled in the art. For example, while exemplary embodiments of the inventive system have been discussed for illustrative purposes, it should be understood that the elements described may be constantly updated and improved by technological advances. Accordingly, the scope of the present invention should be considered in terms of the following claims and is understood not to be limited to the details of structure, operation or process steps as shown and described in the specification and drawings.

What is claimed is:

1. An underarm product and package combination comprising:
(a) an underarm product, said underarm product being a liquid, said underarm product being clear; and
(b) a package, said package capable of containing and dispensing said underarm product, said package having an overflow volume of no greater than 118 milliters, said package being clear.

2. The underarm product and package combination according to claim 1, wherein said package is made of plastic.

3. The package according to claim 2, wherein plastic is selected from the group consisting of polyolefins, polyesters, nylon, vinyl, acrylic, polycarbonates, polystyrene, and/or polyeurothane.

4. The underarm product and package combination according to claim 1, wherein said product is a single-phase solution.

5. The underarm product and package combination according to claim 1, wherein said product is a multi-phase solution.

6. The underarm product and package combination according to claim 1, wherein said product is free of suspended or dispersed solids.

7. The underarm product and package combination according to claim 1, wherein said package is a pressurized contact dispenser.

8. The underarm product and package combination according to claim 1, wherein said package is a pressurized non-contact dispenser.

9. The underarm product and package combination according to claim 1, wherein said package having a single layer wall.

10. The underarm product and package combination according to claim 1, wherein said package having a multiple layer wall.
